(19) United States
(12) Patent Application Publication
(10) Pub. No.: US 2025/0095830 A1
(43) Pub. Date: Mar. 20, 2025

(54) SYSTEM AND METHOD FOR MATCHING HEALTHCARE PROVIDERS TO PATIENTS

(71) Applicant: Daniel Morgan, Orlando, FL (US)

(72) Inventor: Daniel Morgan, Orlando, FL (US)

(21) Appl. No.: 18/517,824

(22) Filed: Nov. 22, 2023

Related U.S. Application Data
(60) Provisional application No. 63/538,981, filed on Sep. 18, 2023.

(51) Int. Cl. G16H 40/20; G06Q 30/018; G06Q 40/08; G16H 10/60; G16H 80/00

(52) U.S. Cl. CPC G16H 40/20; G06Q 30/018; G06Q 40/08; G16H 10/60; G16H 80/00

(57) ABSTRACT

A system for matching healthcare providers to patients is disclosed, including at least one user computing device in operable connection with a user network. An application server is in operable communication with the user network to host an application program for matching healthcare provider to patients. The application program includes a user interface module for providing access to the application program via the at least one user computing device. A processor and non-transitory, computer readable memory stores instructions that, when executed by the processor upon a user selection, cause the device to enable the input of patient information. A patient module determines at least one patient clinical parameter and determines, via a healthcare provider allocation module, at least one healthcare provider capable of performing one or more procedures associated with the at least one patient clinical parameter.

### CROSS-REFERENCE TO RELATED APPLICATIONS

The present application claims priority to U.S. Provisional Application No. 63/538,961 filed Sep. 18, 2023, titled “SYSTEM AND METHOD FOR MATCHING HEALTHCARE PROVIDERS TO PATIENTS” which is hereby incorporated by reference in its entirety.

### TECHNICAL FIELD

The embodiments disclosed herein generally relate to computerized systems and methods for matching healthcare providers to patients.

### BACKGROUND

The healthcare industry involves many complex processes which were historically performed manually by patients and providers alike. Patients must decide on a healthcare provider based on various factors including provider specialties, clinical capabilities, cost, insurance coverage, among many others.

Some current systems generate referrals of patient data to receiving providers. This data is often based on geographic location which means many patients go to the accepting providers, however the patient often returns the hospital because clinical needs were not able to be met at the receiving entity. Further, there is no current way to easily know provider clinical capabilities.

The current process for authorizations involves, first, waiting for a provider to accept the patient. Next, clinical information is sent to the insurance company. Authorization often requires many days which must be received before care is provided. Further, calculating a cost of performing care often requires manual input and communication with the patient followed by the manual execution of a contract. Ancillary services are contracted manually on a need basis, however, there is no way to track the status of the service.

### SUMMARY OF THE INVENTION

This summary is provided to introduce a variety of concepts in a simplified form that is further disclosed in the detailed description of the embodiments. This summary is not intended for determining the scope of the claimed subject matter.

The embodiments provided herein disclose a system for matching healthcare providers to patients, including at least one user computing device in operable connection with a user network. An application server is in operable communication with the user network to host an application program for matching healthcare provider to patients. The application program includes a user interface module for providing access to the application program via the at least one user computing device. A processor and non-transitory, computer readable memory stores instructions that, when executed by the processor upon a user selection, cause the device to enable the input of patient information. A patient module determines at least one patient clinical parameter and determines, via a healthcare provider allocation module, at least one healthcare provider capable of performing one or more procedures associated with the at least one patient clinical parameter.

The embodiments provide an efficient and accurate means of allocating patients to a care provider as well as various other processes involved in the clinic to patient interaction. This may include drafting and executing contracts, monitoring contracts, monitoring patient interactions with the healthcare clinic (including treatment progress), automating the cost of patient care, payment integration, insurance integration, and other resources.

In one aspect, a communication module permit the transmission of patient information to a third-party.

In one aspect, the communication module permits the transmission of patient information to an insurance provider.

In one aspect, the communication module permits the transmission of patient information to a healthcare facility.

In one aspect, a matching module efficiently matches a patient to a healthcare provider.

In one aspect, the matching module references a clinical capability list to determine if the healthcare provider is capable of each clinical procedure associated with the patient's treatment protocols.

In one aspect, a referral module is in operable communication with the communication module.

In one aspect, the referral module transmits a patient to a third-party healthcare provider based on the clinical capability list.

In one aspect, the referral module references an insurance provider list to determine if the patient qualifies for a healthcare provider based on the patient's insurance coverage.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a system architecture diagram of the network infrastructure, according to some embodiments; and

FIG. 2 illustrates a block diagram of the application program and computing system, according to some embodiments;

FIG. 3 illustrates a screenshot of the user dashboard, according to some embodiments;

FIG. 4 illustrates a screenshot of the permissions interface, according to some embodiments;

FIG. 5 illustrates a screenshot of the contract creation interface, according to some embodiments; and

FIG. 6 illustrates a screenshot of the contract interface, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments herein relate to a web-based application which facilitates patient information collection, processing, and referrals based on the patient's clinical information. Information is transmitted to the appropriate level of care providers using provider clinical capability in real time. The system also allows for contracts to be generated and a care cost for and outpatients anticipated care for those needing care without a payer source. The system may also suggest resources to eliminate discharge and determine any potential throughput barriers. Finally, the platform provides a means for generating and transmitting for private agreements, public contracts, and insurance authorizations with real-time monitoring of the same.

In some embodiments, the system is in communication with healthcare providers (i.e., hospitals, and similar care facilities) and connects with medical record systems. The system communicates the healthcare entities which will be receiving information through our platform and will fill out a clinical capability list detailing which scope of care they can perform. Once the hospital or clinic case manager has decided who needs to be referred to another entity, they log into the application program and type in the patient's name and identifying information (e.g., DOB, social security number, and/or medical record number). Once the system identifies a match based on the patient's name and/or identifying information, a read-only copy of the current clinical record is transmitted to the database. The case manager may then decide to which type of level of care entity the referral needs to go to. The case manager may refer to a skilled nursing facility (nursing home), a home health service, assisted living facility, dialysis center, physician telehealth service, etc. Next the case manager will select a list of clinical services that the patient will need from the clinical capability list. Based on the services manually selected, the referral will filter out all of the appropriate levels of care that cannot meet the patient clinical need within the selected geographic location. Once enough data is collected, the system automates the referral process based on the key words provided in the patient chart.

In some embodiments, the system may allow end users to send patient referrals (necessary clinical information) from one healthcare entity to another healthcare provider based on current patient clinical status pairing with clinical capability of the receiving healthcare provider within a geographic location.

In some embodiments, the system may be in communication with other non-provider community entities (i.e., third-parties) that will provide select services that will greatly assist with complex patient discharge root causes. The case manager may select the service needed and the read only referral (patient information) will be sent specifically to the community entity appropriate for the patient. These third-parties will also have access to the application program and may review patient documents to make a decision whether it is a factsheet or clinical diagnosis. This allows for these third-parties to start processing the patient while they are still in the hospital, clear the barriers which allow the patients to be placed safely in healthcare providing entities.

In some embodiments, the third-parties may include law firms, consultants, physician review extensions, among other patient placement specialists.

In some embodiments, the system allows insurance companies and other payors to also receive their own patient information in real time, so that care can be authorized quickly to the receiving healthcare provider.

Each receiving healthcare providers may fill out a list of insurance provider and/or insurance plans which they accept. The system is in communication various insurance companies and payers. Each insurance company and payer may receive limited access to the application program for designated personnel who are in charge of reviewing incoming medical information while deciding and authorizing care specifically for their members. For patients who are uploaded into the system and have a payor, the case manager will select the type of insurance company or benefit from a list of drop down options in the platform. The partnered insurance companies will receive the patient information through the platform in real time and may then review and authorize the care. There will be an option for them to upload the contract to pay for the services at the level they decide is necessary for their client based on the patient chart.

The application program may utilize the known average cost of care from various healthcare providers. Based on the costs attributed to various services, the system will allocate a number that will allow for a small profit while keeping costs low for private payers.

For example, if physical therapy typically costs a nursing home $25/day to provide. Anytime that service is selected it will charge the hospital $37/day if a patient needs the service. Once a hospital or clinic has decided that they will bear the cost of the patients care so that they can discharge them from their facility to make room for paying patients, they will upload a patient from their EMR. Next, they will generate a contract which pulls up the clinical capability tab and allows them to select from the list of a la carte clinical services that the patient will need. Once they have finished selecting, the final recommended number is shown in the contract that will be referred to the healthcare provider through the application program. This eliminates case managers from having to guess how much to offer, also it protects private payors from being taken advantage of all the while ensuring that the receiving healthcare provider will be able to make a profit of off the future care that is given.

In some embodiments, the system is able to track and monitor private agreements, public contracts, and insurance documents executed between a third party and the patient. Each document is stored in the systems database once uploaded. The payment authorizer may select the amount of time that the agreement is valid which will trigger the timeline in the electronic calendar to track the dates of current contracts. The other party then has the opportunity to sign digitally or manually and reupload to the database once properly executed. Once a contract has been executed it stays in the calendar until completion of the terms discussed in the contract.

Henceforth, any party can sign in and look at current contracts at any time, with real time tracking for all current contracts within an organization. The search can be further broken down by location (if one company has multiple locations), by patient name, or by individual authorizer. After the contract has been completed, it will be stored indefinitely within the database as long as the company partnered continues to use the service and remain in good standing.

In some embodiments, the system may provide notifications at or near the expiration of such agreements. However, while a contract is still current, a director level access to the application program from either party (referring healthcare or receiving healthcare or ancillary service) can set their notification preferences so that they would be notified about potentially expiring contracts or agreements. The user may elect to receive an email with a link to the contract that's expiring for follow up, and/or if the user has it downloaded to their phone, they can receive a push notification (like a txt message, or general notification etc.).

The recommended determination for an extension on private pay contracts will be based on if ending current contracted services will not result in a readmission back to the hospital following that clinical level of care. This is also done through the notification system and the if the receiving healthcare provider elects to extend the contract or request clinical/medical review. The provider would upload pertinent documents for the physician to review and then a recommended determination will be made and notified to the referring healthcare entity so they can decide if they would like to continue paying post recommended time or end the contract at the calendar date initially set.

In the case in which patients require more resources to assist with the long term or complex aspect of a contract, the app will provide real time ancillary providers to remove barriers from placement and potential future readmissions.

In some embodiments, receiving entities have real time clinical information from the referring EMR allowing transparency and confidence in accepting patients, thus resulting in faster response times.

With established payment methods (contracts, authorizations agreements) linked to the patient referral in real time, the patient spends less time (2+ days) in the hospital waiting for a receiving provider to accept with guaranteed payment. This lowers the hospital bill for patients and insurance companies and also allows the hospital or clinic to have the bed ready to provide care to another waiting individual, granting bed turnover efficiency and lowers length of stay.

With the contracts being tracked, both entities know where their money is coming from and going which may limit billing discrepancies. Finally, it allows upper level management to generate trends and budget appropriately for the services they provide and needs they can maximize.

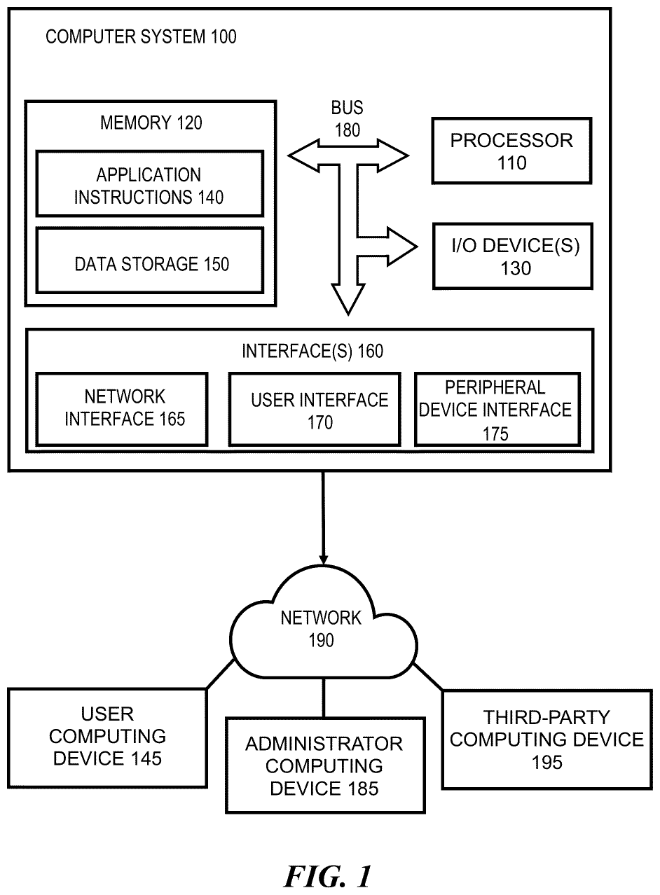

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C #, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized a user (e.g., a healthcare provider) to interact with the various functionalities of the system including to perform patient rounds, handoff patient rounding responsibility, perform biometric verification tasks, and other associated tasks and functionalities of the system. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

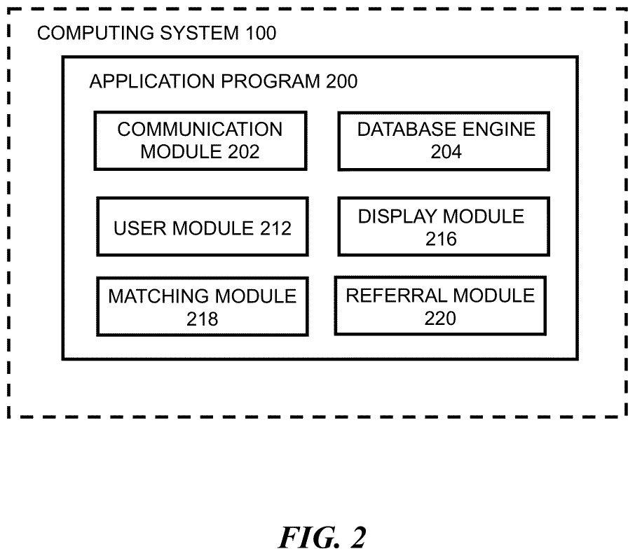

FIG. 2 illustrates an example computer architecture for the application program 200 operated via the computer system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a communication module 202, a database engine 204, a user module 212, a display module 216, a patient matching module 218, and a referral module 220.

In some embodiments, the communication module 202 is configured for receiving, processing, and transmitting a user command and/or one or more data streams. In such embodiments, the communication module 202 performs communication functions between various devices, including the user computing device 145, the administrator computing device 185, and a third-party computing device 195. In some embodiments, the communication module 302 is configured to allow one or more users of the system, including a third-party, to communicate with one another. In some embodiments, the communications module 202 is configured to maintain one or more communication sessions with one or more servers, the administrative computing device 185, and/or one or more third-party computing device(s) 195.

In some embodiments, the communication module 202 allows for the transmission patient information, insurance provider information, third-party information, and the like. This information is used by the system in the various ways described hereinabove.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations.

The database engine 214 may be utilized to transmit patient information, insurance provider information, third-party information, and the like.

The user module 212 may store user preferences including the user account information, historical usage data, user personal information, patient information, third party information and the like.

In some embodiments, the display module 216 is configured to display one or more graphic user interfaces, including, e.g., one or more user interfaces, one or more consumer interfaces, one or more video presenter interfaces, etc. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored. The display module 216 provides alerts to the user device which can be viewed and acknowledged by the user.

In some embodiments, the matching module 218 receives a plurality of patient information and utilizes a comparator to compare the patient information with healthcare provider information. The healthcare provider information may include clinical capabilities, scheduling information, insurance compatibility, etc.

In some embodiments, the matching module 218 matches the patient to the healthcare provider based on financial status (i.e., is the patient able to pay for the clinical need). If this is not met, a payer is created. A healthcare entity may send a referral based on clinical needs or financial status. The matching module 218 may filter through who accepts the payer and which healthcare entity provides the clinical service to select for which individuals meet both criteria. If the clinical need is met, but the financial need is not met, the healthcare provider may become the payer using the application program.

In some embodiments, the referral module 220 is in operable communication with the communication module 202 to permit the communication between the patient, physician, healthcare provider, third-party, etc. to accurately refer a patient to a suitable care provider.

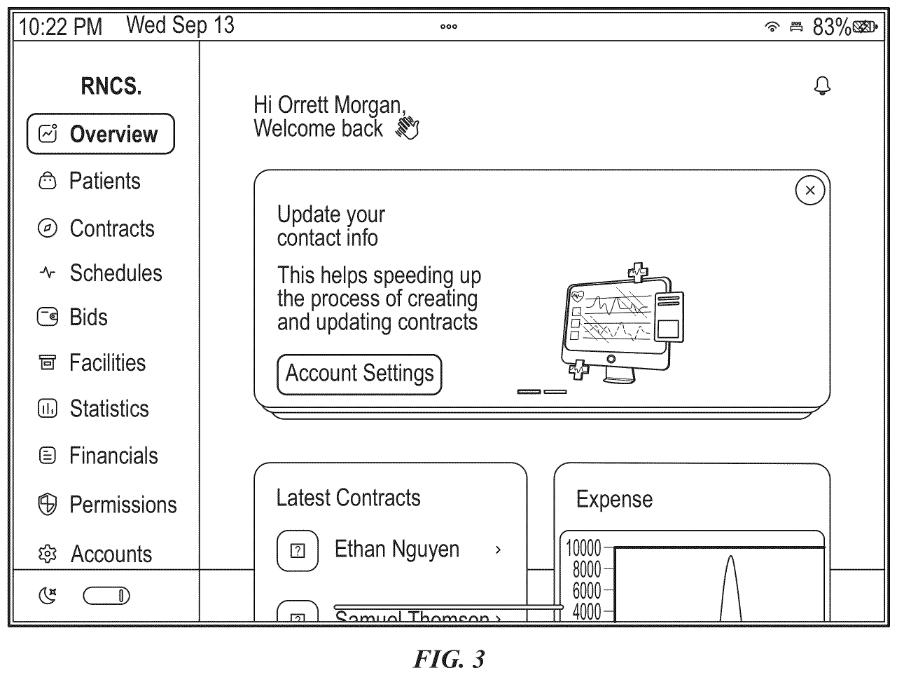

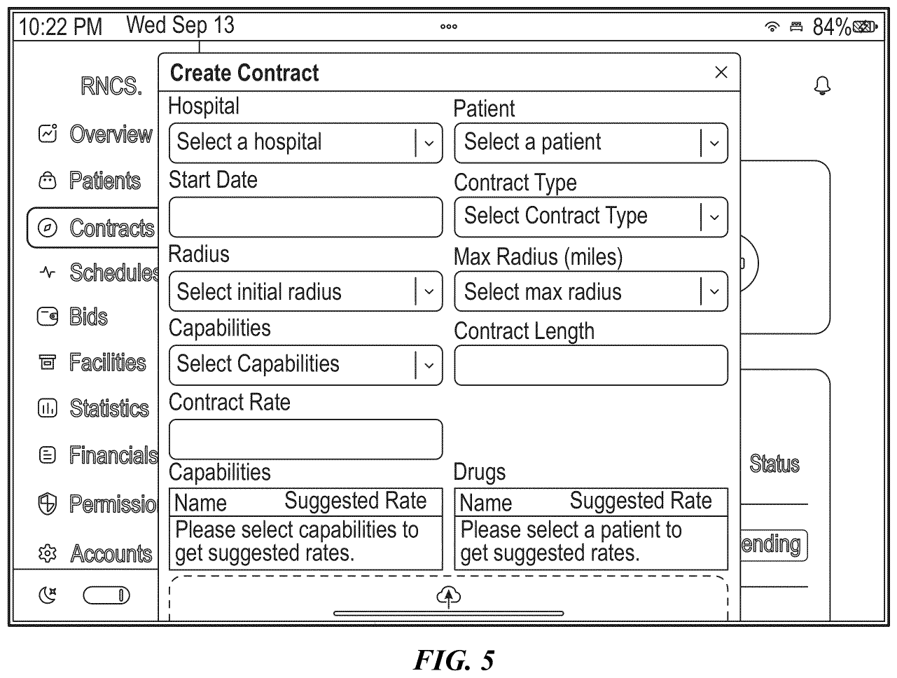

FIGS. 3-6 illustrate exemplary user interfaces for the system. One skilled in the arts will readily understand that the ornamental features of the user interface may change while retaining the inventive functionalities described hereinabove. FIG. 3 illustrates a screenshot of the user dashboard. This user dashboard may include a patients tab, contracts tab, schedules tab, bids tab, facilities tab, statistics tab, financials tab, and permissions tab. Users may select to view previous contracts, or create a new contract as shown in FIG. 5.

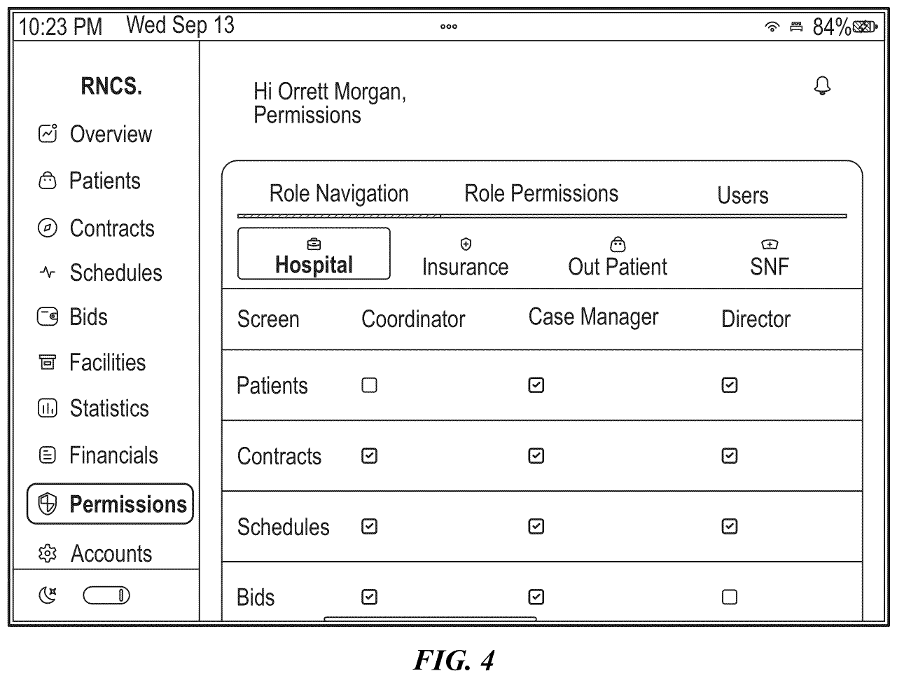

FIG. 4 illustrates a screenshot of the permissions interface wherein the user may establish user roles, permissions for each role, and add/edit user information. The user may also indicate a facility (e.g., a hospital) associated with the user, insurance information, out-patient information, etc. Users may be identified as a coordinator, case manager, director, etc. as their role.

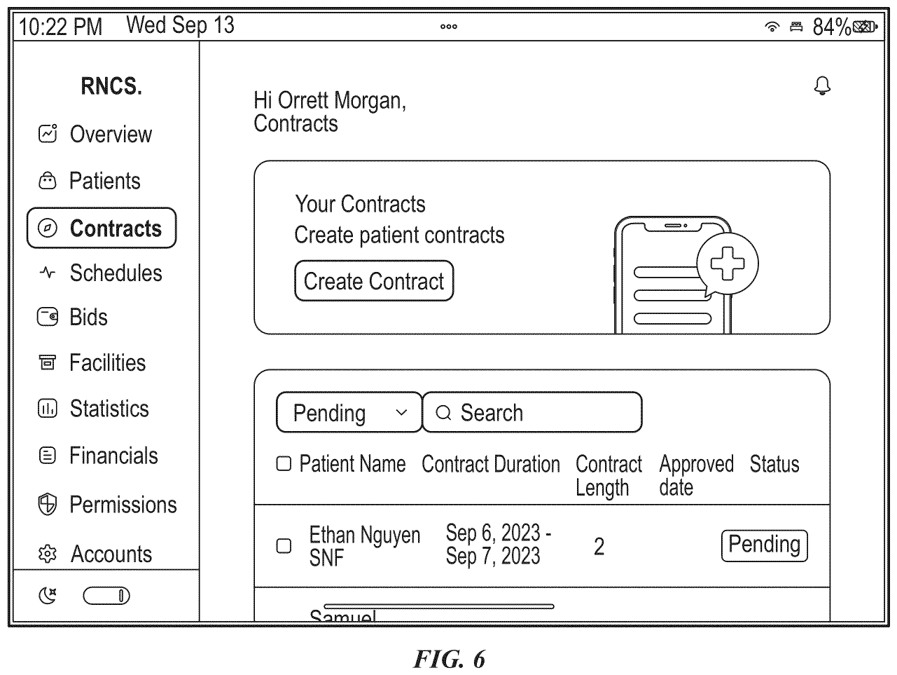

FIG. 5 illustrates the contract creation interface. The contract creation interface includes a hospital selection menu, start date input field, radius for the facility search, capabilities of the facility, contract rate, patient selection, contract type, contract length, and other useful information which is used to create the contract. FIG. 6 illustrates a screenshot of the contract interface wherein the user may select from various contract to view information associated therewith.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of illustrating, explaining, and describing embodiments of this disclosure. Modifications and adaptations to these embodiments will be apparent to those skilled in the art and may be made without departing from the scope or spirit of this disclosure.

As used herein and in the appended claims, the singular forms “a”, “an”, and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. A system for matching healthcare providers to patients, the system comprising:
at least one user computing device in operable connection with a user network;
an application server in operable communication with the user network, the application server configured to host an application program for matching healthcare provider to patients, the application program having a user interface module for providing access to the application program via the at least one user computing device;
a processor and a non-transitory, computer readable memory storing instructions that, when executed by the processor upon a user selection, cause the device to:
enable the input of patient information;
determine, via a patient module, at least one patient clinical parameter;
determine, via a healthcare provider allocation module, at least one healthcare provider capable of performing one or more procedures associated with the at least one patient clinical parameter.

2. The system of claim 1, further comprising a communication module to permit the transmission of patient information to a third-party.

3. The system of claim 2, wherein the communication module permits the transmission of patient information to an insurance provider.

4. The system of claim 2, wherein the communication module permits the transmission of patient information to a healthcare facility.

5. The system of claim 1, further comprising a matching module to efficiently match a patient to a healthcare provider.

6. The system of claim 5, wherein the matching module references a clinical capability list to determine if the healthcare provider is capable of each clinical procedure associated with the patient's treatment protocols.

7. The system of claim 1, further comprising a referral module in operable communication with the communication module.

8. The system of claim 7, wherein the referral module transmits a patient to a third-party healthcare provider based on the clinical capability list.

9. The system of claim 8, wherein the referral module references an insurance provider list to determine if the patient qualifies for a healthcare provider based on the patient's insurance coverage.

10. A system for matching healthcare providers to patients, the system comprising:
at least one user computing device in operable connection with a user network;
an application server in operable communication with the user network, the application server configured to host an application program for matching healthcare provider to patients, the application program having a user interface module for providing access to the application program via the at least one user computing device;
a processor and a non-transitory, computer readable memory storing instructions that, when executed by the processor upon a user selection, cause the device to:
enable the input of patient information;
determine, via a matching module, at least one patient clinical parameter and determine if the patient is suitable for one or more healthcare providers, wherein the matching module determines if the patient meets a financial status;
refer, via a referral module, the patient to the one or more healthcare providers based on the at least one clinical parameter; and
a user module to store a plurality of patient information utilized by the matching module and referral module.

11. The system of claim 10, further comprising a communication module to permit the transmission of patient information to a third-party.

12. The system of claim 11, wherein the communication module permits the transmission of patient information to an insurance provider.

13. The system of claim 12, wherein the communication module permits the transmission of patient information to a healthcare facility.

14. The system of claim 13, further comprising a matching module to efficiently match a patient to a healthcare provider.

15. The system of claim 14, wherein the matching module references a clinical capability list to determine if the healthcare provider is capable of each clinical procedure associated with the patient's treatment protocols.

16. The system of claim 15, further comprising a referral module in operable communication with the communication module.

17. The system of claim 16, wherein the referral module transmits a patient to a third-party healthcare provider based on the clinical capability list.

18. The system of claim 17, wherein the referral module references an insurance provider list to determine if the patient qualifies for a healthcare provider based on the patient's insurance coverage.

19. The system of claim 18, further comprising a contract creation interface to provide means for inputting a plurality of contract information to create a contract.

20. A method for matching healthcare providers to patients, the method comprising the steps of:
a processor and a non-transitory, computer readable memory storing instructions that, when executed by the processor upon a user selection, cause the device to perform the following:
enabling the input of patient information using a contract creation interface;
determining, via a matching module, at least one patient clinical parameter and determine if the patient is suitable for one or more healthcare providers, wherein the matching module determines if the patient meets a financial status;
referring, via a referral module, the patient to the one or more healthcare providers based on the at least one clinical parameter; and
storing a plurality of patient information utilized by the matching module and referral module.
